(19) United States
(12) Patent Application Publication
(10) Pub. No.: US 2025/0109416 A1
(43) Pub. Date: Apr. 3, 2025

(54) METHOD FOR PRODUCING 3-PHENYLPROPIONIC ACID ANALOGUE COMPOUNDS

(71) Applicant: MARUZEN PHARMACEUTICALS CO., LTD., Onomichi-shi, Hiroshima (JP)

(72) Inventors: Riyo AWA, Fukuyama-shi, Hiroshima (JP); Yosuke NISHITANI, Fukuyama-shi, Hiroshima (JP)

(21) Appl. No.: 18/834,312

(22) PCT Filed: Jan. 31, 2023
(86) PCT No.: PCT/JP2023/003055

§ 371 Date: Jul. 30, 2024

(30) Foreign Application Priority Data
Jan. 31, 2022 (JP) 2022-013560

(51) Int. Cl. C12P 7/52; C07C 53/134; C07C 57/44; C07C 59/11; C07C 59/52; C12N 1/20; C12R 1/01

(52) U.S. Cl. CPC C12P 7/52; C07C 53/134; C07C 57/44; C07C 59/11; C07C 59/52; C12N 1/205; C07C 2601/16; C12R 2001/01

(57) ABSTRACT

Provided is a method for producing a 3-phenylpropionic acid analogue compound represented by Formula (I), the method comprising a step of culturing lactic acid bacteria of genus Weissella using a culture medium that contains 3 mM or more of a cinnamic acid analogue compound represented by Formula (II), the lactic acid bacteria having properties (1a) to (3a) when inoculated to an MRS liquid culture medium containing 3 mM 4-hydroxy-3-methoxycinnamic acid and cultured at 37° C. for 24 hours under an anaerobic condition: (1a) a turbidity of 1.0 or more; (2a) a ratio of reduction to 3-(4-hydroxy-3-methoxyphenyl)propionic acid of 80% or more; and (3a) a ratio of decarboxylation to 4-vinylguaiacol of less than 5%. Also provided are lactic acid bacteria having these properties. The above method and lactic acid bacteria allow a 3-Phenylpropionic acid analogue compound to be efficiently produced.

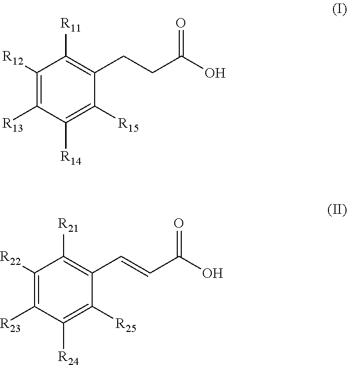

### TECHNICAL FIELD

The present invention relates to a method for producing a 3-phenylpropionic acid analogue compound and, in more detail, to a method for efficiently producing a 3-phenylpropionic acid analogue compound from a cinnamic acid analogue compound as a raw material using lactic acid bacteria. The present invention also relates to lactic acid bacteria for which the above method for production can be carried out.

### BACKGROUND ART

In recent years, lifestyle-related diseases such as obesity, type 2 diabetes, hypertension, and insulin resistance have become major issues due to lifestyle habits such as overeating and lack of physical activity. In order to improve these lifestyle-related diseases, attention is being paid not only to treatment with pharmaceuticals, but also to prevention/treatment using ingredients derived from natural products or foods.

Compounds of 3-Phenylpropionic acid analogues are recognized as having an action of playing a role in such lifestyle-related diseases, and their functionality has attracted attention. For example, it has been reported that 3-(4-hydroxy-3-methoxyphenyl)propionic acid has an inhibitory action on cAMP phosphodiesterase activity, an inhibitory action on dipeptidyl peptidase IV activity, etc. (see Patent Document 1).

It has been revealed that Lactobacillus plantarum, lactic acid bacteria isolated from pickles or the like, have two pathways for decomposing cinnamic acid analogue compounds contained in plant cell walls, such as 4-hydroxy-3-methoxycinnamic acid. For example, when 4-hydroxy-3-methoxycinnamic acid, a type of cinnamic acid analogue compounds, is added to a culture medium and cultured, 3-(4-hydroxy-3-methoxyphenyl)propionic acid, which is a reduction product of the propenoic acid portion of 4-hydroxy-3-methoxycinnamic acid, and 4-vinylguaiacol, which is a decarboxylation product of 4-hydroxy-3-methoxycinnamic acid, are detected.

The present inventors have revealed that when 4-hydroxy-3-methoxycinnamic acid is added into and cultured in a culture medium from which dissolved oxygen has been removed, the reduction reaction takes precedence over the decarboxylation reaction and 3-(4-hydroxy-3-methoxyphenyl)propionic acid is preferentially produced (see Patent Document 2), and have also identified 4-hydroxy-3-methoxycinnamic acid reductase (see Patent Document 3).

### PRIOR ART DOCUMENTS

### Patent Documents

[Patent Document 1] JP2016-079186A
[Patent Document 2] JP2019-017381A
[Patent Document 3] JP2021-137010A

### SUMMARY OF THE INVENTION

### Problems to be Solved by the Invention

Objects of the present invention include providing a method for production that can efficiently produce a 3-phenylpropionic acid analogue compound and providing lactic acid bacteria useful for the method for production.

### Means for Solving the Problems

As a result of intensive studies to solve the above problems, the present inventors have found that by using lactic acid bacteria of the genus Weissella that have certain properties, a 3-phenylpropionic acid analogue compound can be produced more efficiently than by conventional methods, and have thus accomplished the present invention.

Specifically, the present invention is as follows.

<1> A method for producing a 3-phenylpropionic acid analogue compound represented by Formula (I) below,

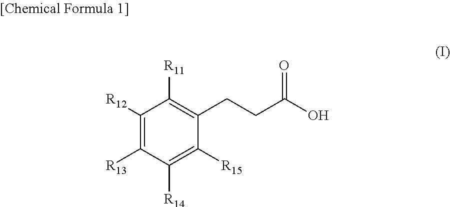

(in Formula (I), R₁₁ to R₁₅ are each independently a hydrogen atom, a hydroxy group, an alkoxy group whose carbon number is 1 to 3, or an alkyl group whose carbon number is 1 to 3)
the method comprising a step of culturing lactic acid bacteria of genus Weissella using a culture medium that contains 3 mM or more of a cinnamic acid analogue compound represented by Formula (II) below,

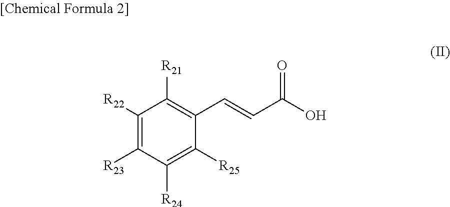

(in Formula (II), R₂₁ to R₂₅ are identical with corresponding R₁₁ to R₁₅ in the above Formula (I), respectively)
the lactic acid bacteria having properties (1a) to (3a) below when inoculated to an MRS liquid culture medium containing 3 mM 4-hydroxy-3-methoxycinnamic acid and cultured at 37° C. for 24 hours under an anaerobic condition:
(1a) a turbidity of 1.0 or more;
(2a) a ratio of reduction to 3-(4-hydroxy-3-methoxyphenyl)propionic acid of 80% or more; and
(3a) a ratio of decarboxylation to 4-vinylguaiacol of less than 5%.
   <2> The method for production according to <1>, wherein the lactic acid bacteria are Weissella cibaria or Weissella confusa.
   <3> The method for production according to <1> or <2>, wherein the lactic acid bacteria belong to Weissella cibaria 011YN2 strain (accession number: NITE BP-03579), Weissella cibaria 054YN2 strain (accession number: NITE BP-03578), or Weissella confusa NRBC3957 strain.
   <4> The method for production according to any one of <1> to <3>, wherein the lactic acid bacteria have properties (1b) to (3b) below when inoculated to an MRS liquid culture medium containing 5 mM 4-hydroxy-3-methoxycinnamic acid and cultured at 37° C. for 24 hours under an anaerobic condition:
(1b) a turbidity of 1.0 or more;
(2b) a ratio of reduction to 3-(4-hydroxy-3-methoxyphenyl)propionic acid of 80% or more; and
(3b) a ratio of decarboxylation to 4-vinylguaiacol of less than 5%.
   <5> Lactic acid bacteria of genus Weissella having properties (1a) to (3a) below when inoculated to an MRS liquid culture medium containing 3 mM 4-hydroxy-3-methoxycinnamic acid and cultured at 37° C. for 24 hours under an anaerobic condition:
(1a) a turbidity of 1.0 or more;
(2a) a ratio of reduction to 3-(4-hydroxy-3-methoxyphenyl)propionic acid of 80% or more; and
(3a) a ratio of decarboxylation to 4-vinylguaiacol of less than 5%.
   <6> Lactic acid bacteria of genus Weissella having properties (1b) to (3b) below when inoculated to an MRS liquid culture medium containing 5 mM 4-hydroxy-3-methoxycinnamic acid and cultured at 37° C. for 24 hours under an anaerobic condition:
(1b) a turbidity of 1.0 or more;
(2b) a ratio of reduction to 3-(4-hydroxy-3-methoxyphenyl)propionic acid of 80% or more; and
(3b) a ratio of decarboxylation to 4-vinylguaiacol of less than 5%.
   <7> The lactic acid bacteria according to <5> or <6>, wherein the lactic acid bacteria are Weissella cibaria or Weissella confusa.
   <8> The lactic acid bacteria according to any one of <5> to <7>, wherein the lactic acid bacteria belong to Weissella cibaria 011YN2 strain (accession number: NITE BP-03579) or Weissella cibaria 054YN2 strain (accession number: NITE BP-03578).

### Advantageous Effect of the Invention

According to the method of the present invention, a 3-phenylpropionic acid analogue compound can be produced efficiently. Moreover, the lactic acid bacteria according to the present invention are useful for the above method for production.

### EMBODIMENTS FOR CARRYING OUT THE INVENTION

Hereinafter, one or more embodiments of the present invention will be described.

### <Lactic Acid Bacteria>

The lactic acid bacteria according to one embodiment of the present invention are lactic acid bacteria of the genus Weissella, which, when inoculated to a liquid culture medium containing a high concentration of 4-hydroxy-3-methoxycinnamic acid (which may be abbreviated as HMCA, hereinafter) and cultured under an anaerobic condition, grow sufficiently and have properties of a sufficiently high ratio of reduction to 3-(4-hydroxy-3-methoxyphenyl)propionic acid (which may be abbreviated as HMPA, hereinafter) and a sufficiently low ratio of decarboxylation to 4-vinylguaiacol (which may be abbreviated as 4VG, hereinafter).

The inventors have found that if the HMCA concentration is high during the process of screening lactic acid bacteria useful for producing HMPA, the growth of lactic acid bacteria of the genus Weissella tends to be inhibited depending on the strain, or, even though they grow, the ratio of reduction to HMPA tends to decrease.

In contrast, among the strains capable of reducing HMCA to HMPA, if the strain can grow sufficiently even in a high concentrations of HMCA (e.g., 3 mM or more), the reaction of reducing HMCA to HMPA is promoted as the strain grows, and HMPA can therefore be produced more efficiently. Furthermore, if the strain has a sufficiently low ratio of decarboxylation to 4VG, HMCA is not consumed more than necessary, and HMPA can be produced further efficiently.

The inventors have confirmed that 4-hydroxy-3-methoxycinnamate reductase identified in Lactobacillus plantarum (see Patent Document 3) is not found in lactic acid bacteria of the genus Weissella, and it is believed that the reduction reaction of HMCA by lactic acid bacteria of the genus Weissella is due to an enzyme different from that of Lactobacillus plantarum.

### (Culture Conditions)

The lactic acid bacteria according to the present embodiment are specified by the properties when they are cultured under a predetermined culture condition. The predetermined culture condition refers to a condition in which the lactic acid bacteria are inoculated to a liquid culture medium containing a high concentration of HMCA and cultured under an anaerobic condition.

The liquid culture medium used under the above culture condition may be any culture medium that can be used to culture lactic acid bacteria, and examples of such a culture medium include MRS culture medium and GYP culture medium. Among these, MRS culture medium is preferred. The MRS culture medium specifically has the following composition.

TABLE 1

| | | |
|---|---|---|
| Peptone | 10 | g |
| Beef extract | 10 | g |
| Yeast extract | 5 | g |
| Glucose | 20 | g |
| Tween 80 | 1 | g |
| K₂HPO₄ | 2 | g |
| Sodium acetate | 5 | g |
| Diammonium hydrogen | 2 | g |
| cit rate | | |
| MgSO₄•7H₂O | 0.2 | g |
| MnSO₄•nH₂O | 0.05 | g |
| Distilled water | 1 | L |

The concentration of HMCA under the above culture condition may be a high concentration, specifically 3 mM or more, and examples of the concentration include 3 mM, 5 mM, 10 mM, and 50 mM.

The above predetermined culture condition refers to a case of an initial turbidity (OD₆₆₀) of 0.01 at the time of inoculation, an anaerobic condition, and culturing at 37° C. for 24 hours.

The “anaerobic condition” in the present embodiment may be, for example, a state in which the dissolved oxygen concentration in the liquid culture medium is lower than a saturated state (e.g., about 6.7 mg/L at 37° C.). For example, the dissolved oxygen concentration in the liquid culture medium may be 6 mg/L or less in an embodiment, 5 mg/L or less in another embodiment, 3 mg/L or less in still another embodiment, 1 mg/L or less in yet another embodiment, 0.5 mg/L or less in still yet another embodiment, or 0.2 mg/L or less in yet still another embodiment.

Methods for achieving such an anaerobic condition are not particularly limited. For example, a culture medium having a low dissolved oxygen concentration can be prepared through heat sterilizing (pressurizing) the liquid culture medium to remove dissolved oxygen so as not to come into contact with oxygen such as that of air and then immediately sealing the liquid culture medium with an airtight stopper or the like to maintain the state without contact with oxygen. Other known methods can also be adopted, such as an anaerobic jar method, an anaerobic bag method, a nitrogen substitution method, a steel wool method, and other similar methods for reducing the oxygen concentration in the gas phase thereby to reduce the dissolved oxygen concentration in the culture medium; and methods for reducing the dissolved oxygen concentration by adding additives such as deoxidant and antioxidant to the liquid culture medium.

In order to maintain the above anaerobic condition during culture, it is preferred that the culture medium during culture should not be in contact with the gas phase or should be in contact with the gas phase in which the oxygen concentration is maintained lower than that of air.

### (Properties of Lactic Acid Bacteria)

The lactic acid bacteria according to the present embodiment, when cultured under the above culture condition, have the following properties (1) to (3):
(1) the lactic acid bacteria grow sufficiently;
(2) the ratio of reduction from HMCA to HMPA is sufficiently high; and
(3) the ratio of decarboxylation from HMCA to 4VG is sufficiently low.

Here, the above (1) the lactic acid bacteria grow sufficiently specifically means that the turbidity (OD₆₆₀) after 24 hours of culture under the above culture condition is a predetermined value or more. The turbidity after 24 hours of culture can be set, for example, to 1.0 or more in an embodiment, 1.2 or more in another embodiment, or 1.5 or more in still another embodiment.

The above (2) the ratio of reduction from HMCA to HMPA is sufficiently high specifically means that the ratio of reduction to HMPA after 24 hours of culture under the above culture condition is a predetermined value or more. The ratio of reduction to HMPA after 24 hours of culture can be set, for example, to 80% or more in an embodiment, 85% or more in another embodiment, 90% or more in still another embodiment, or 95% or more in yet another embodiment.

The above (3) the ratio of decarboxylation from HMCA to 4VG is sufficiently low specifically means that the ratio of decarboxylation to 4VG after 24 hours of culture under the above culture condition is less than a predetermined value. The ratio of decarboxylation to 4VG after 24 hours of culture can be set, for example, to less than 5% in an embodiment, less than 4% in another embodiment, or less than 3% in still another embodiment.

The above predetermined culture condition and the above properties (1) to (3) can be set, for example, as follows.

When the lactic acid bacteria are inoculated to a liquid culture medium containing 3 mM HMCA and cultured at 37° C. for 24 hours under an anaerobic condition, the following properties (1a) to (3a) are possessed:
(1a) the turbidity is 1.0 or more;
(2a) the ratio of reduction to HMPA is 80% or more; and
(3a) the ratio of decarboxylation to 4VG is less than 5%.

Additionally or alternatively, the above predetermined culture condition and the above properties (1) to (3) may be set, for example, as follows.

When the lactic acid bacteria are inoculated to a liquid culture medium containing 5 mM HMCA and cultured at 37° C. for 24 hours under an anaerobic condition, the following properties (1b) to (3b) are possessed:
(1b) the turbidity is 1.0 or more;
(2b) the ratio of reduction to HMPA is 80% or more; and
(3b) the ratio of decarboxylation to 4VG is less than 5%.

As the HMCA concentration increases, the growth of lactic acid bacteria of the genus Weissella tends to be inhibited, or the ratio of reduction to HMPA tends to decrease even though the lactic acid bacteria grow. In other words, it can be said that if the lactic acid bacteria have the above properties (1b) to (3b) when the initial HMCA concentration is 5 mM, they will have the above properties (1a) to (3a) when the initial HMCA concentration is 3 mM.

### (Types of Lactic Acid Bacteria/Method of Acquisition)

The lactic acid bacteria used in the present embodiment are not particularly limited, provided that they are lactic acid bacteria of the genus Weissella having the above properties.

Examples of lactic acid bacteria of the genus Weissella include Weissella cibaria, Weissella confusa, Weissella hellenica, and Weissella oryzae. Any of these may be used, but Weissella cibaria or Weissella confusa may be preferred.

When using Weissella cibaria, the Weissella cibaria 011YN2 strain (accession number: NITE BP-03579) or Weissella cibaria 054YN2 strain (accession number: NITE BP-03578) used in the examples to be described later can be exemplified as a particularly suitable strain. These strains were isolated from pickles by the present inventors and have been deposited at the National Institute of Advanced Industrial Science and Technology/International Patent Organism Depositary.

When using Weissella confusa, the Weissella confusa NBRC 3957 strain can be exemplified as a suitable strain. The NBRC 3957 strain is preserved at the National Institute of Technology and Evaluation, Biological Resource Center, and is listed in the NBRC catalog as a distributable strain.

The lactic acid bacteria used in the present embodiment can be obtained by a known screening method. For example, the lactic acid bacteria of the present embodiment can be obtained through culturing a specimen of Weissella lactic acid bacteria under the previously described culture condition and determining whether or not the specimen satisfies the above properties (1) to (3).

In such screening, the lactic acid bacteria may be cultured in advance in a solid culture medium or the like containing a high concentration of HMCA thereby to preliminarily screen the lactic acid bacteria as to whether or not they grow sufficiently (corresponding to the above property (1)), and then a determination may be made whether or not the above properties (1) to (3) are satisfied in a liquid culture medium. As referred to herein, the “high concentration” is the same as that defined for the liquid culture medium used when determining the above properties (1) to (3).

Furthermore, by repeating culture in a culture medium containing a high concentration of HMCA, it is possible to enrich the lactic acid bacteria of the genus Weissella with improved HMCA production efficiency. For example, if there are lactic acid bacteria of the genus Weissella that have the above properties (1a) to (3a) when the initial HMCA concentration is 3 mM, but do not have the above properties (1b) to (3b) when the initial HMCA concentration is 5 mM, then, by repeatedly culturing the lactic acid bacteria in a culture medium containing 5 mM or more HMCA, strains of the lactic acid bacteria that are able to efficiently utilize HMCA can be selected. Among the strains selected in this way, there are strains that have come to satisfy the above properties (1b) to (3b). Therefore, by determining whether or not the selected strains satisfy the above properties (1b) to (3b), it is possible to obtain lactic acid bacteria of the genus Weissella with improved HMCA production efficiency.

The above-described lactic acid bacteria of the genus Weissella can efficiently reduce HMCA to HMPA and therefore are particularly useful for the production of HMPA and, in turn, for the production of a 3-phenylpropionic acid analogue compound.

### <Method for Producing 3-Phenylpropionic Acid Analogue Compound>

A method for producing a 3-phenylpropionic acid analogue compound according to one embodiment of the present invention includes a step of culturing the lactic acid bacteria of the genus Weissella according to the above embodiment using a culture medium containing a high concentration of a cinnamic acid analogue compound.

The 3-phenylpropionic acid analogue compound produced in the present embodiment is a compound represented by the following Formula (I) (which may be simply referred to as “3-phenylpropionic acid or acids,” hereinafter).

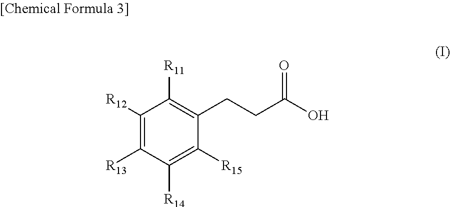

In the above Formula (I), R₁₁ to R₁₅ are each independently a hydrogen atom, a hydroxy group, an alkoxy group whose carbon number is 1 to 3, or an alkyl group whose carbon number is 1 to 3.

Among these, a hydrogen atom, a hydroxy group, and a methoxy group are preferred. In particular, R₁₁, R₁₄, and R₁₅ are preferably hydrogen atoms, and R₁₂ and R₁₃ are preferably each independently a hydrogen atom, a hydroxy group, or a methoxy group.

Compounds particularly preferred as the 3-phenylpropionic acids produced by the method of the present embodiment are exemplified as follows.

HMPA: R₁₁═H, R₁₂═OCH₃, R₁₃═OH, R₁₄═H, R₁₅═H

3-Phenylpropionic acid: R₁₁═H, R₁₂═H, R₁₃═H, R₁₄═H, R₁₅═H

3-(3, 4-dihydroxyphenyl) propionic acid: R₁₁═H, R₁₂═OH, R₁₃═OH, R₁₄═H, R₁₅═H

3-(4-hydroxyphenyl) propionic acid: R₁₁═H, R₁₂═H, R₁₃═OH, R₁₄═H, R₁₅═H

In the method for production of the present embodiment, the cinnamic acid analogue compound used as a raw material is a compound represented by the following Formula (II) (which may be simply referred to as “cinnamic acid or acids,” hereinafter).

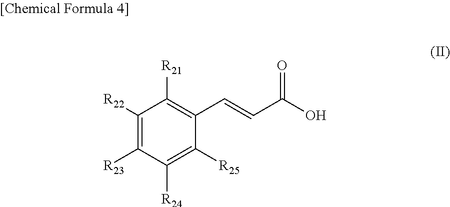

In the above Formula (II), R₂₁ to R₂₅ are identical with corresponding R₁₁ to R₁₅ in the above Formula (I), respectively.

Here, R₁₁ to R₁₅ in Formula (I) and R₂₁ to R₂₅ in Formula (II) that have the same substitution positions are referred to as “corresponding.” For example, what corresponds to R₁₁ in Formula (I) is R₂₁ in Formula (II).

The lactic acid bacteria of the genus Weissella used in the present embodiment can efficiently reduce HMCA to HMPA, but as demonstrated in the examples to be described later, the lactic acid bacteria can also reduce cinnamic acids other than HMCA. The reason for this is thought to be that the HMCA reductase of the lactic acid bacteria of the genus Weissella has a relatively tolerant substrate specificity with respect to substituent groups of the benzene rings of cinnamic acids. The above lactic acid bacteria of the genus Weissella can therefore reduce cinnamic acids other than HMCA and can also be used in a reaction for producing 3-phenylpropionic acids other than HMPA.

For cinnamic acids as the raw materials, in principle, compounds in which R₂₁ to R₂₅ in Formula (II) are identical with the corresponding R₁₁ to R₁₅, respectively, in the compounds of Formula (I) (3-phenylpropionic acids) to be produced may be used.

Here, the culture medium used in the step of culturing the lactic acid bacteria of the genus Weissella is not particularly limited, provided that it is a culture medium that can be used for culturing lactic acid bacteria. For example, the culture medium may be the same as the liquid culture medium for specifying the properties of the lactic acid bacteria in the above embodiment or may also be a different culture medium. Examples of such a culture medium include MRS culture medium and GYP culture medium. A culture medium containing a plant material or the like that is available as a food material can also be suitably used, and when such a culture medium is used, the obtained culture solution can be used as a food material or the like without any modification.

The pH of the above culture medium is preferably 2 to 9 and more preferably 3 to 8. The culture temperature is preferably 20° C. to 45° C., more preferably 25° C. to 40° C., and particularly preferably 30° C. to 38° C. The pH and culture temperature of the culture medium within such ranges are suitable for the growth of the lactic acid bacteria of the genus Weissella, and the ratio of reduction from cinnamic acids to 3-phenylpropionic acids may be desirable.

The concentration of cinnamic acids in the culture medium used in the above step can be preferably 3 mM or more in an embodiment, 5 mM or more in another embodiment, 10 mM or more in still another embodiment, 20 mM or more in yet another embodiment, 50 mM or more in still yet another embodiment, or 100 mM or more in yet still another embodiment, and can be appropriately set in consideration of the above properties (1) to (3) of the lactic acid bacteria of the genus Weissella to be used. By increasing the concentration of cinnamic acids, a culture having a high concentration of 3-phenylpropionic acids can be obtained in this step.

Here, the cinnamic acids used in this step may be purified products or compositions containing cinnamic acids. Examples of compositions containing cinnamic acids include extracts of plants or the like containing cinnamic acids. Examples of such plants include crushed materials and extracts of plants such as rice, wheat, barley, corn, bamboo, coffee, tomato, mate, mugwort, and burdock. Since cinnamic acids are components of lignin in woody plants and herbaceous plants, lignin or a composition containing it may also be used.

Among these, rice, barley, bamboo (bamboo shoots), etc. are preferred from the viewpoints of ease of availability, cinnamic acid content, etc., and rice or rice bran is particularly preferred.

The culture in this step is preferably performed under an anaerobic condition. Since the lactic acid bacteria of the genus Weissella are facultative anaerobic bacteria, culturing under an anaerobic condition is suitable for the growth of the lactic acid bacteria of the genus Weissella. In addition, culturing under an anaerobic condition allows the ratio of reduction from cinnamic acids to 3-phenylpropionic acids to be desirable.

The meaning of an anaerobic condition and the method of achieving an anaerobic condition are as described in the previously described embodiment. Unlike the previously described embodiment, the anaerobic condition in this step may not necessarily be strictly controlled. Therefore, even if an anaerobic condition is not satisfied in early stages of the culture, for example, the culture condition may become an anaerobic condition as a result of the consumption of dissolved oxygen in association with the growth of the lactic acid bacteria of the genus Weissella.

The culture obtained in this culture step contains a high concentration of 3-phenylpropionic acids. Depending on the subsequent purpose, the culture obtained may be used as a food material without any modification, or may be appropriately subjected to treatment for decolorization, clarification, etc. Examples of such treatment include activated carbon treatment and resin treatment. The above culture may also be made into a composition with an increased concentration of 3-phenylpropionic acids by fractionation/purification or the like. Methods for fractionation/purification are not particularly limited, and known means such as column chromatography, HPLC, and recrystallization can be appropriately adopted.

According to the above-described method for producing 3-phenylpropionic acids, the lactic acid bacteria of the genus Weissella having the above properties can be cultured using a culture medium containing a high concentration of cinnamic acids, thereby producing 3-phenylpropionic acids in high yields.

The embodiments heretofore explained are described to facilitate understanding of the present invention and are not described to limit the present invention. It is therefore intended that the elements disclosed in the above embodiments include all design changes and equivalents to fall within the technical scope of the present invention.

### EXAMPLES

Hereinafter, the present invention will be described in more detail by exemplifying testing examples, etc., but the present invention is not limited in the following testing examples, etc.

### «Testing Example 1» Culture Test-1

The lactic acid bacteria listed in Table 2 were used and cultured in MRS culture medium containing HMCA, and the turbidity, the residual ratio of HMCA, the formation ratio of HMPA, and 4VG were measured as follows.

The lactic acid bacteria used in this test are as follows. The 011YN2 and 054YN2 strains were isolated from pickles by the present inventors, and were identified as lactic acid bacteria belonging to the Weissella cibaria by determining the 16S rDNA base sequence according to an ordinary method.

TABLE 2

| Species name | Strain name | Source |
|---|---|---|
| Weissella cibaria | JCM7777 | RIKEN BRC-JCM |
| | 011YN2 | Isolated from pickles |
| | 054YN2 | Isolated from pickles |
| Weissella confusa | NBRC3955 | NITE NBRC |
| | NBRC3957 | NITE NBRC |
| | NBRC3958 | NITE NBRC |
| | NBRC106469^{T} | NITE NBRC |

RIKEN BRC-JCM: Institute of Physical and Chemical Research, BioResource Research Center, Microbe Division/Japan Collection of Microorganisms
NITE NBRC: National Institute of Technology and Evaluation, Biological Resource Center

In this culture test, MRS culture medium (available from Merck) was used to culture the lactic acid bacteria, and the pH was adjusted to 7.5. HMCA available from Tokyo Chemical Industry Co., Ltd. was used. Anaeropack for anaerobic culture (available from Mitsubishi Gas Chemical Company, Inc.) was used for the anaerobic condition.

The bacteria grown on MRS agar culture medium were inoculated to MRS liquid culture medium and pre-cultured under an anaerobic condition at 37° C. for 24 hours.

The obtained pre-culture solution was adjusted in the MRS liquid culture medium to have a turbidity (OD₆₆₀) of 1.0, and 1 mL of the adjusted pre-culture solution was inoculated to 99 mL of MRS liquid culture medium containing each concentration of HMCA (initial turbidity (OD₆₆₀): 0.01, final HMCA concentration: see Tables 3 to 5) and cultured under an anaerobic condition at pH 7.5 and 37° C. for 24 hours.

After 24 hours, the turbidity (OD₆₆₀) of the culture solution was measured, and the concentrations of HMCA, HMPA, and 4-vinylguaiacol (4VG) in the culture solution were analyzed by HPLC. The standards used were HMCA (available from Tokyo Chemical Industry Co., Ltd.), HMPA (available from Tokyo Chemical Industry Co., Ltd.), and 4VG (available from Alfa Aesar).

The obtained HMCA, HMPA, and 4VG concentrations (all in mM) were divided by the initial HMCA concentration to calculate the residual ratio of HMCA, the ratio of reduction to HMPA, and the generation ratio of 4VG, respectively.

The results are listed in Tables 3 to 5.

### =HPLC Condition=

Column: Wakosil II 5C18HG φ4.6 mm×250 mm
Column temperature: 40° C.
Injection volume: 10 μL
Mobile phases: A solution: 0.1% TFA aqueous solution, B solution: acetonitrile
   0 min-12 min: Mixture of A and B solutions (82:18)
   12 min-20 min: Mixture of A and B solutions (82:18)-(75:25)
   20 min-35 min: Mixture of A and B solutions (75:25)-(70:30)
   35 min-40 min: Mixture of A and B solutions (70:30)
   40 min-50 min: Mixture of A and B solutions (10:90)
   50 min-65 min: Mixture of A and B solutions (82:18)
Flow rate: 0 min-40 min: 1.0 mL/min
   40 min-55 min: 1.4 mL/min
   55 min-65 min: 1.0 mL/min
Detector: UV detector
Detection wavelength: HMPA, 4VG: 280 nm
HMCA: 320 nm

TABLE 3
Initial HMCA concentration: 1 mM

| | | Residual | Ratio of | Generation | |
|---|---|---|---|---|---|
| Species | | ratio of | reduction | ratio of | |
| name | Strain name | HMCA | to HMPA | 4VG | Turbidity |
| Weissella | JCM7777 | 98% | 0% | 0% | 3.06 |
| cibaria | 011YN2 | 0% | 99% | 0% | 1.88 |
| | 054YN2 | 0% | 100% | 0% | 3.14 |
| Weissella | NBRC 3955 | 99% | 0% | 0% | 3.12 |
| confusa | NBRC 3957 | 1% | 99% | 0% | 2.75 |
| | NBRC 3958 | 99% | 0% | 0% | 2.45 |
| | NBRC106469 ^{T} | 98% | 0% | 0% | 3.57 |

TABLE 4
Initial HMCA concentration: 3 mM

| | | Residual | Ratio of | Generation | |
|---|---|---|---|---|---|
| Species | | ratio of | reduction | ratio of | |
| name | Strain name | HMCA | to HMPA | 4VG | Turbidity |
| Weissella | JCM7777 | 98% | 0% | 0% | 2.99 |
| cibaria | 011YN2 | 0% | 98% | 0% | 1.80 |
| | 054YN2 | 0% | 100% | 0% | 3.09 |
| Weissella | NBRC 3955 | 99% | 0% | 0% | 3.00 |
| confusa | NBRC 3957 | 17% | 83% | 0% | 2.68 |
| | NBRC 3958 | 99% | 0% | 0% | 2.42 |
| | NBRC106469 ^{T} | 98% | 0% | 0% | 3.49 |

TABLE 5
Initial HMCA concentration: 5 mM

| | | Residual | Ratio of | Generation | |
|---|---|---|---|---|---|
| Species | | ratio of | reduction | ratio of | |
| name | Strain name | HMCA | to HMPA | 4VG | Turbidity |
| Weissella | JCM7777 | 98% | 0% | 0% | 2.90 |
| cibaria | 011YN2 | 0% | 100% | 0% | 3.05 |
| | 054YN2 | 0% | 100% | 0% | 1.76 |
| Weissella | NBRC 3955 | 99% | 0% | 0% | 3.01 |
| confusa | NBRC 3957 | 92% | 5% | 0% | 2.50 |
| | NBRC 3958 | 99% | 0% | 0% | 2.52 |
| | NBRC106469 ^{T} | 98% | 0% | 0% | 2.87 |

As the inventors' knowledge, it is found that when the HMCA concentration increases, the growth of lactic acid bacteria tends to be inhibited depending on the strain, or the ratio of reduction to HMPA tends to decrease even though the lactic acid bacteria grow. It has been revealed that by using lactic acid bacteria of the genus Weissella that satisfy the requirements of the present invention, HMCA can be efficiently reduced to HMPA even at high concentrations (3 mM or more) of HMCA. On the other hand, strains that were not able to reduce HMCA to HMPA even at an initial HMCA concentration of 1 mM (such as JCM7777 strain) were not able to reduce HMCA to HMPA also at 3 mM or 5 mM.

### «Testing Example 2» Culture Test-2

Weissella cibaria 011YN2 and 054YN2 strains were used to carry out a culture test in a jar fermenter.

In this culture test, MRS culture medium (available from Merck) was used to culture the lactic acid bacteria. Rice bran extract (available from TSUNO FOOD INDUSTRIAL CO., LTD.) (HMCA content 80 mass %) was used as the HMCA source. A 2 L jar fermenter was used for culture. The dissolved oxygen concentration was measured during culture and confirmed to be maintained at 0.2 mg/L or less.

The bacteria grown on the MRS agar culture medium were inoculated to MRS liquid culture medium containing 50 mM HMCA and pre-cultured at 37° C. for 24 hours under an anaerobic condition. The obtained pre-culture solution 20 mL was inoculated to 2 L of MRS liquid culture medium (HMCA: 50 mM) filled in the above jar fermenter and cultured at 37° C.

The concentrations of HMCA and HMPA were measured 24 hours after inoculation to the jar fermenter in the same manner as in Testing Example 1. The results are listed in Table 6.

TABLE 6

| | | | Residual | Ratio of | Generation | |
|---|---|---|---|---|---|---|
| Species | Strain | HMCA | ratio of | reduction | ratio of | |
| name | name | concentration | HMCA | to HMPA | 4VG | Turbidity |
| Weissella | 011YN2 | 50 mM | 0% | 99% | 0% | 3.52 |
| cibaria | 054YN2 | 50 mM | 0% | 98% | 0% | 3.02 |

### «Testing Example 3» Culture Test-3

Weissella cibaria 054YN2 strain was used to carry out a culture test in a jar fermenter.

This culture test was conducted in the same manner as in Testing Example 2 except that the conditions for adding the rice bran extract as the HMCA source were modified. Between 0 and 12 hours after the start of culture in the jar fermenter, rice bran extract was added to a total of 63 g (HMCA addition concentration: 130 mM) or a total of 131 g (HMCA addition concentration: 270 mM). The results are listed in Table 7.

TABLE 7

| | | HMCA | Residual | Ratio of | Generation | |
|---|---|---|---|---|---|---|
| Species | Strain | addition | ratio of | reduction | ratio of | |
| name | name | concentration | HMCA | to HMPA | 4VG | Turbidity |
| Weissella | 054YN2 | 130 mM | 0% | 98% | 0% | 2.85 |
| cibaria | 054YN2 | 270 mM | 0% | 97% | 0% | 2.90 |

### «Testing Example 4> Culture Test-4

Weissella cibaria 054YN2 strain was used to test the production of 3-phenylpropionic acids other than HMPA.

In this culture test, MRS culture medium (available from Merck) was used to culture the lactic acid bacteria, and cinnamic acid and caffeic acid (both available from FUJIFILM Wako Pure Chemical Corporation) were used as the cinnamic acids. Anaeropack for anaerobic culture (available from Mitsubishi Gas Chemical Company, Inc.) was used for the anaerobic condition.

The bacteria grown on MRS agar culture medium were inoculated to MRS liquid culture medium and pre-cultured under an anaerobic condition at 37° C. for 24 hours. The obtained pre-culture solution 1 mL was inoculated to 99 mL of MRS liquid culture medium containing cinnamic acids (see Table 9 for the type and final concentration of cinnamic acids) and cultured under an anaerobic condition at pH 7.5 and 37° C. for 24 hours.

After 24 hours, the turbidity (OD₆₆₀) of the culture solution was measured, and for the cinnamic acids and 3-phenylpropionic acids (PPAs), which are reduced products of the cinnamic acids, respective concentrations in the culture solution were analyzed by HPLC. The HPLC condition was the same as in Testing Example 1, and the standards and detection wavelengths were as listed in Table 8.

TABLE 8

| | | | Detection |
|---|---|---|---|
| Cinnamic | | | wavelength |
| acids | Compound name | Source | (nm) |
| Cinnamic | Cinnamic acid | FUJIFILM Wako Pure | 280 |
| acids | | Chemical | |
| | | Corporation | |
| | Caffeic acid | FUJIFILM Wako Pure | 320 |
| | | Chemical | |
| | | Corporation | |
| PPAS | 3-Phenylpropionic acid | FUJIFILM Wako Pure | 210 |
| | (PPA) | Chemical | |
| | | Corporation | |
| | 3-(3,4-dihydroxyphenyl ) | FUJIFILM Wako Pure | 280 |
| | phenylpropionic acid | Chemical | |
| | | Corporation | |

The obtained concentrations of cinnamic acids and the concentrations of PPAs (both in mM) were divided by the initial concentrations of cinnamic acids to calculate the residual ratio of cinnamic acids and the ratio of reduction to PPAs, respectively.

The results are listed in Table 9.

TABLE 9

| | | Residual | | |
|---|---|---|---|---|
| | Concentration | ratio of | Ratio of | |
| Cinnamic | of cinnamic | cinnamic | reduction | |
| acids | acids | acids | to PPAs | Turbidity |
| Cinnamic | 5 mM | 1% | 99% | 1.71 |
| acid | 50 mM | 6% | 93% | 1.70 |
| Caffeic | 5 mM | 1% | 98% | 1.65 |
| acid | | | | |

As listed in Table 9, the lactic acid bacteria of the genus Weissella were able to reduce cinnamic acids other than HMCA. It has thus confirmed that the lactic acid bacteria of the genus Weissella of the present invention can also be used to produce 3-phenylpropionic acids other than HMPA.

1. A method for producing a 3-phenylpropionic acid analogue compound represented by Formula (I) below, 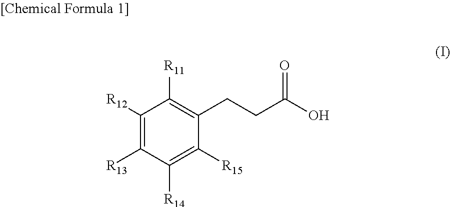
(in Formula (I), R₁₁ to R₁₅ are each independently a hydrogen atom, a hydroxy group, an alkoxy group whose carbon number is 1 to 3, or an alkyl group whose carbon number is 1 to 3)
the method comprising a step of culturing lactic acid bacteria of genus Weissella using a culture medium that contains 3 mM or more of a cinnamic acid analogue compound represented by Formula (II) below, 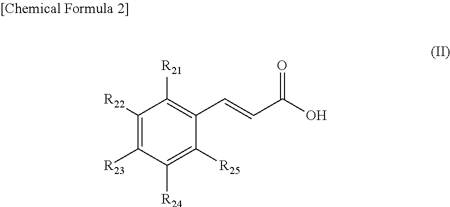
(in Formula (II), R₂₁ to R₂₅ are identical with corresponding R₁₁ to R₁₅ in the above Formula (I), respectively)
the lactic acid bacteria having properties (1a) to (3a) below when inoculated to an MRS liquid culture medium containing 3 mM 4-hydroxy-3-methoxycinnamic acid and cultured at 37° C. for 24 hours under an anaerobic condition:
(1a) a turbidity of 1.0 or more;
(2a) a ratio of reduction to 3-(4-hydroxy-3-methoxyphenyl)propionic acid of 80% or more; and
(3a) a ratio of decarboxylation to 4-vinylguaiacol of less than 5%.

2. The method for production according to claim 1, wherein the lactic acid bacteria are Weissella cibaria or Weissella confusa.

3. The method for production according to claim 1, wherein the lactic acid bacteria belong to Weissella cibaria 011YN2 strain (accession number: NITE BP-03579), Weissella cibaria 054YN2 strain (accession number: NITE BP-03578), or Weissella confusa NRBC3957 strain.

4. The method for production according to claim 1, wherein the lactic acid bacteria have properties (1b) to (3b) below when inoculated to an MRS liquid culture medium containing 5 mM 4-hydroxy-3-methoxycinnamic acid and cultured at 37° C. for 24 hours under an anaerobic condition:
(1b) a turbidity of 1.0 or more;
(2b) a ratio of reduction to 3-(4-hydroxy-3-methoxyphenyl)propionic acid of 80% or more; and
(3b) a ratio of decarboxylation to 4-vinylguaiacol of less than 5%.

5. Lactic acid bacteria of genus Weissella having properties (1a) to (3a) below when inoculated to an MRS liquid culture medium containing 3 mM 4-hydroxy-3-methoxycinnamic acid and cultured at 37° C. for 24 hours under an anaerobic condition:
(1a) a turbidity of 1.0 or more;
(2a) a ratio of reduction to 3-(4-hydroxy-3-methoxyphenyl)propionic acid of 80% or more; and
(3a) a ratio of decarboxylation to 4-vinylguaiacol of less than 5%.

6. Lactic acid bacteria of genus Weissella having properties (1b) to (3b) below when inoculated to an MRS liquid culture medium containing 5 mM 4-hydroxy-3-methoxycinnamic acid and cultured at 37° C. for 24 hours under an anaerobic condition:
(1b) a turbidity of 1.0 or more;
(2b) a ratio of reduction to 3-(4-hydroxy-3-methoxyphenyl)propionic acid of 80% or more; and
(3b) a ratio of decarboxylation to 4-vinylguaiacol of less than 5%.

7. The lactic acid bacteria according to claim 5, wherein the lactic acid bacteria are Weissella cibaria or Weissella confusa.

8. The lactic acid bacteria according to claim 5, wherein the lactic acid bacteria belong to Weissella cibaria 011YN2 strain (accession number: NITE BP-03579) or Weissella cibaria 054YN2 strain (accession number: NITE BP-03578).
